(19) United States
(12) Patent Application Publication
(10) Pub. No.: US 2010/0233282 A1
(43) Pub. Date: Sep. 16, 2010

(54) DEVICE AND METHODS FOR DELIVERY OF BIOACTIVE MATERIALS TO THE RIGHT SIDE OF THE HEART

(76) Inventor: Allan Mishra, Menlo Park, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/722,934

(22) Filed: Mar. 12, 2010

Related U.S. Application Data
(60) Provisional application No. 61/159,966, filed on Mar. 13, 2009.

(51) Int. Cl. A61K 35/16; A61M 25/10

(52) U.S. Cl. 424/530; 604/509; 604/523

(57) ABSTRACT

A method of delivering a bioactive composition to the right side of the heart is described. The method can be used to treat cardiac dysfunction such as myocardial infarction, arrhythmias or congestive heart failure. A dual lumen catheter is described for delivering the bioactive substance by inserting a first cannula of a dual lumen catheter into a vein to access the right ventricle. The first cannula has a first balloon which is blown up proximal to the tricuspid valve. A second cannula is then introduced which accesses the right side of the heart and coronary sinus. The bioactive substance is then delivered to the right side of the heart. The bioactive substance may include platelets and/or white blood cells at concentrations higher than what is normally found in whole blood.

### CROSS-REFERENCE TO RELATED APPLICATIONS

This application claims priority to U.S. Provisional Application No. 61/159,966 filed Mar. 13, 2009 which is incorporated herein by reference.

### BACKGROUND

1. Field

A device and methods for delivery of blood products, in particular platelet-rich plasma to heart muscle via the cardiac coronary sinus for treatment of cardiac dysfunction including myocardial infarction, arrhythmia and congestive heart failure is described.

2. Description of the Related Art

The coronary sinus is located between the left atrium and ventricle on the posterior aspect of the heart. It drains venous coronary blood into the right atrium immediately superior to the tricuspid valve. In presently available devices, ten percent of the time a complication occurs when attempting to enter the coronary sinus. Prolonged attempts or even failure to enter the vein often occur.

### SUMMARY

Embodiments are directed to delivering bioactive composition to heart using a dual lumen catheter by inserting a first cannula of a dual lumen catheter into a vein to access the right ventricle. The first cannula has a first balloon which is blown up around the first cannula proximal to the tricuspid valve. A second cannula of the dual lumen catheter is inserted. The second cannula has a second balloon proximal to the tip of the second cannula. The bioactive composition is delivered to the coronary sinus by inflation and then release of the second balloon.

Embodiments also relate to a dual lumen catheter having a first lumen with a first port for accessing the pulmonary valve and a second lumen with a one or more ports for accessing the coronary sinus. The first lumen and second lumen have a dial guide. In some embodiments, the dial guide is the first lumen surrounded by the multiple ports of the second lumen.

Embodiments relate to methods for delivering a bioactive composition to the right side of the heart. Preferably, the bioactive composition includes platelet-rich plasma.

### BRIEF DESCRIPTION OF THE DRAWINGS

FIG. 1 is a schematic anterior partial cutaway view of the right side of the heart showing the dual lumen catheter described herein placed for delivery of bioactive substance.

FIG. 2 shows a cross-sectional view of the dial guide for the catheter of FIG. 1.

### DETAILED DESCRIPTION OF THE PREFERRED EMBODIMENT

A procedure and apparatus are described for introduction of bioactive substances, such as unactivated platelet-rich plasma, to the right side of the heart, preferably the cardiac coronary sinus for treatment of cardiac dysfunction including myocardial infarction, arrhythmia and congestive heart failure.

Specific examples of introducing bioactive materials such as platelet rich plasma into the coronary sinus for cardiac regeneration are also disclosed. Other growth factors, cells, inorganic or organic substances may be placed via this catheter, either singly or in combination. The initial example outlines introduction via the jugular vein. Venous access is preferred via neck or groin. Other applications may use arterial or other access.

### Overview

The term “PRP” as used herein is a broad term which is used in its ordinary sense and is a concentration of platelets greater than the peripheral blood concentration suspended in a solution of plasma. While normal platelet counts may range from about 140,000 to about 400,000 per microliter, some platelet concentrations of PRP may be in the range of about 500,000 to about 1,200,000 per cubic millimeter or more, and some platelet concentrations may be as low as 50,000 per cubic millimeter. PRP may be formed from whole blood, and may be obtained using autologous, allogenic, or pooled sources of platelets and/or plasma. PRP may be formed from a variety of animal sources, including human sources. In some examples, PRP may be further processed, including but not limited to leukoreduction and immunoadsorbtion. Other PRP compositions are further described in U.S. Pat. No. 6,811,777, filed Apr. 11, 2003, which is hereby incorporated herein by reference in its entirety.

Platelets actively extrude the growth factors involved in initiating wound healing. These growth factors, also called cytokines, are small proteins each of about 25,000 Daltons molecular weight. They are stored in a granules in platelets. In response to platelet to platelet aggregation or platelet to connective tissue contact, the cell membrane of the platelet is “activated” to release these alpha granules. These growth factors include platelet derived growth factors (PDGF), transforming growth factor beta 1 and 2 (TGF-β), fibronectin, vitronectin, fibrin and insulin-like growth factor (ILGF). These growth factors function to assist the body in repairing itself by stimulating stem cells to regenerate new tissue and by promoting vascularization.

In preferred embodiments, the PRP composition does not include an exogenous activator of PRP. Typical agents that are used to exogenously activate platelets are thrombin, epinephrine, collagen, calcium salts, adenosine diphosphate, and arachidonic acid.

Certain embodiments relate to where the platelet composition is at or above physiological pH. Preferably, the platelet-rich plasma composition is buffered to a pH of 7.3 to 7.5. Certain embodiments also relate to a method wherein the platelet composition comprises platelets obtained from the patient.

The PRP composition can be made and then stored in a frozen or lyophilized state to be applied to the tissue later. In a preferred form it would be buffered to physiologic pH but it may also be valuable to instill PRP at either acidic or basic pH for specific clinical indications such as ablation of an abnormal conduction pathway. In yet another embodiment, the PRP could be prepared in a form that is depleted of neutrophils or other fractions of white blood cells either partially or completely. In some embodiments, the PRP composition contains white blood cells at a concentration higher than baseline levels, preferable 2-10 times baseline levels, more preferably, 3-5 times baseline levels of white blood cells. In some embodiments, the PRP composition is depleted of neutrophils, preferably 10-50% of the neutrophils present in the baseline composition, more preferably less than 10% of the neutrophils present in the baseline composition, most preferably, less than 1% of the level present in the baseline composition. The baseline composition for PRP compositions is understood to be the normal concentrations found in blood from healthy individuals.

The treatments and kits described herein are applicable to both human and non-human animals. The term “patient” as used herein refers to either a human or non-human patient. Particularly preferred applications are for veterinary animals such as horses, pigs, cows, sheep, cats and dogs. Most preferred embodiments are directed to treatment of human patients.

Cardiac dysfunction includes but is not limited to myocardial infarction, congestive heart failure, and arrhythmias.

The term “arrhythmia” is used broadly herein to refer to cardiac abnormalities involving a disturbance in initialization and/or propagation of the impulses in a heart. The disturbance may be localized to a portion of the conduction tissues and/or may affect the entire electrical conduction system of the heart. There are several possible types of arrhythmias of varying severity.

An arrhythmia may be initially detected in a patient as an abnormally fast (i.e., tachycardia) or slow (i.e., bradycardia) heartbeat. Furthermore, some arrhythmias or conduction patterns may be characterized as regular, irregularly irregular (e.g., atrial fibrillation) or regularly irregular (e.g., Wenckebach or second degree heart block—type 1). The specific type of arrhythmia from which a patient may be suffering may be diagnosed based on an electrocardiogram (ECG or EKG). A normal electrocardiogram, as is known, depicts a PQRST-wave. The specific arrhythmia may be diagnosed based on one or more deviations from a normal PQRST-wave.

Myocardial infarction may be identified by determining whether enzymes such as cardiac troponin (e.g., troponin-I or T), creatine kinase (CK) including CK-MB, aspartate transaminase (AST)/Glutamic Oxaloacetic Transaminase (GOT/SGOT)/aspartate aminotransferase (ASAT), lactate dehydrogenase (LDH), and/or myoglobin (Mb), and/or the like are present in the blood stream. The PRP compositions described herein may be delivered in the absence of the enzymes. Myocardial infarctions may be determined by identifying ST elevation in an ECG (e.g., during rest, a pharmacological stress test, and/or a physiological stress test), by coronary angiogram (e.g., noting acute closure of a vessel supplying myocardium at risk), by a nuclear medicine scan (e.g., technetium-99m or thalium-201), etc.

The PRP composition may be delivered to a patient in an emergency situation or as part of an elective procedure. For example, the PRP composition may be delivered in an emergency room to treat a myocardial infarction or ventricular tachycardia. In other instances, the PRP composition may be delivered weeks after an event, such as an arrhythmia, during an elective cardioversion.

Further, according to some embodiments, the PRP composition may comprise PRP and one or more active agents. For example, the active agents may include anti-arrhythmic agents and/or anti-coagulants.

The compositions, devices, methods, and kits described herein are illustrative of various embodiments, variations, and adaptations. The disclosure is not intended to be limited to only the embodiments described.

### Compositions

The PRP composition may comprise a PRP derived from a human or animal source of whole blood. The PRP may be prepared from an autologous source, an allogenic source, a single source, or a pooled source of platelets and/or plasma. To derive the PRP, whole blood may be collected, for example, using a blood collection syringe. The amount of blood collected may depend on a number of factors, including, for example, the amount of PRP desired, the health of the patient, the severity or type of the cardiac dysfunction, the availability of prepared PRP, or any suitable combination of factors. Any suitable amount of blood may be collected. For example, about 20 cc to about 150 cc of blood may be drawn. More specifically, about 27 cc to about 110 cc or about 27 cc to about 55 cc of blood may be withdrawn. In some embodiments, the blood may be collected from a patient who may be presently suffering, or who has previously suffered from, a cardiac dysfunction. PRP made from a patient's own blood may significantly reduce the risk of adverse reactions or infection.

The PRP may be prepared in any suitable way. For example, the PRP may be prepared from whole blood using a centrifuge. The whole blood may or may not be cooled after being collected. Isolation of platelets from whole blood depends upon the density difference between platelets and red blood cells. The platelets and white blood cells are concentrated in the layer (i.e., the “buffy coat”) between the platelet depleted plasma (top layer) and red blood cells (bottom layer). For example, a bottom buoy and a top buoy may be used to trap the platelet-rich layer between the upper and lower phase. This platelet-rich layer may then be withdrawn using a syringe or pipette. Generally, at least 60% or at least 80% of the available platelets within the blood sample can be captured. These platelets may be resuspended in a volume that may be about 3% to about 20% or more preferably about 5% to about 10% of the sample volume. The platelets are preferably suspended in plasma but may alternatively be suspended in buffered aqueous solution or physiological saline.

In an exemplary embodiment, about 55 cc of blood may be withdrawn into a 60 cc syringe (or another suitable syringe) that contains about 5 cc of an anticoagulant, such as a citrate dextrose solution. The syringe may be attached to an apheresis needle, and primed with the anticoagulant. Blood (about 27 cc to about 55 cc) may be drawn from the patient using standard aseptic practice. In some embodiments, a local anesthetic such as anbesol, benzocaine, lidocaine, procaine, bupivicaine, or any appropriate anesthetic known in the art may be used to anesthetize the insertion area.

In some examples, the blood may then be centrifuged using a gravitational platelet system, such as the Cell Factor Technologies GPS System® centrifuge. The blood-filled syringe containing between about 20 cc to about 150 cc of blood (e.g., about 55 cc of blood) and about 5 cc citrate dextrose may be slowly transferred to a disposable separation tube which may be loaded into a port on the GPS centrifuge. The sample may be capped and placed into the centrifuge. The centrifuge may be counterbalanced with about 60 cc sterile saline, placed into the opposite side of the centrifuge. Alternatively, if two samples are prepared, two GPS disposable tubes may be filled with equal amounts of blood and citrate dextrose. The samples may then be spun to separate platelets from blood and plasma. The samples may be spun at about 2000 rpm to about 5000 rpm for about 5 minutes to about 30 minutes. For example, centrifugation may be performed at 3200 rpm for extraction from a side of the separation tube and then isolated platelets may be suspended in about 3 cc to about 5 cc of plasma by agitation. The PRP may then be extracted from a side port using, for example, a 10 cc syringe. If about 55 cc of blood may be collected from a patient, about 5 cc of PRP may be obtained.

The PRP may be buffered using an alkaline buffering agent to a physiological pH. The buffering agent may be a biocompatible buffer such as HEPES, TRIS, monobasic phosphate, monobasic bicarbonate, or any suitable combination thereof that may be capable of adjusting the PRP to physiological pH between about 6.5 and about 8.0. In certain embodiments, the physiological pH may be from about 7.3 to about 7.5, and may be about 7.4. For example, the buffering agent may be an 8.4% sodium bicarbonate solution. In these embodiments, for each cc of PRP isolated from whole blood, 0.05 cc of 8.4% sodium bicarbonate may be added. In some embodiments, the syringe may be gently shaken to mix the PRP and bicarbonate.

As noted above, in preferred embodiments, no exogenous activator is added to the PRP composition. However, in some instances, the PRP composition may comprise one or more additional agents, diluents, solvents, or other ingredients, including an exogenous activator(s). Examples of the additional agents include, but are not limited to, thrombin, epinephrine, collagen, calcium salts, pH adjusting agents, materials to promote degranulation or preserve platelets, additional growth factors or growth factor inhibitors, NSAIDS, steroids, anti-infective agents, and mixtures and combinations of the foregoing.

In some embodiments, anti-arrhythmic agents may be included in the PRP compositions. Such agents are classified using the Vaughan Williams classification. In the Vaughan Williams classification, Class I drugs operate by interfering with the sodium (Na+) channel and include, for example, quinidine, procainamide, disopyramide, lidocaine, phenytoin, mexiletine, tocainide, encainide, flecainide, indecainide, propafenone, and moricizine. Class II agents are beta blockers and include, for example, propranolol, esmolol, timolol, metoprolol, sotalol, and atenolol. Class III agents affect potassium (K+) efflux and include bretylium, amiodarone, sotalol, ibutilide, and dofetilide. Class IV agents affect calcium channels and the AV node and include, for example, verapamil and diltiazem. Class V agents work by other or unknown mechanisms and include, for example, moricizine, digoxin, and adenosine. Any suitable anti-arrhythmic drug and/or combination thereof may be added to the PRP composition. The specific formulation used may be determined based on, for example, the type of arrhythmia, patient history, drug interactions, or any other suitable factor.

Furthermore, the PRP compositions may comprise a contrast agent for detection by an imaging technique such as X-rays, magnetic resonance imaging (MRI), or ultrasound. Examples of such contrast agents include, but are not limited to, X-ray contrast (e.g., IsoVue), MRI contrast (e.g., gadolinium), and ultrasound contrast.

### Device

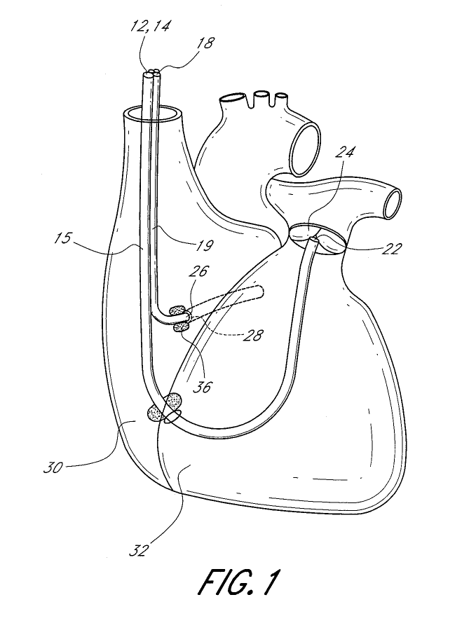

A device useful in the described method is shown in FIG. 1 and includes a dual lumen catheter 10 having a first lumen 12 with a first port 14 for a first cannula 15 and a second lumen 16 with a second port 18 for a second cannula 19.

The length of the first cannula 15 is longer than the length of the second cannula 19. Optionally, the two cannulas 15 and 19 may be bound together over a portion of the length of the second cannula 19. As the first cannula acts more like an anchor and is not used to deliver bioactive material, it may be open or closed.

The cannulas are flexible and made of biocompatible material. The length and diameter is any convenient length. The length is appropriate to point of entry which is preferably venous entry, preferably via groin or vein in the neck. For entry via the jugular vein, the length is conveniently 200-300 cm. Diameter of the cannulas is 0.01 mm or more.

The distal portion 22 of the first cannula 15 is adapted to access the pulmonary valve 24. The distal portion 26 of the second cannula 19 is adapted to access the coronary sinus 28. A balloon 30 covers the tricuspid valve 32. Once the path to the pulmonary valve is blocked by the first cannula and first balloon, the second cannula can more readily access pre-determined sections of the right side of the heart, such as the coronary sinus, for delivery of bioactive compositions.

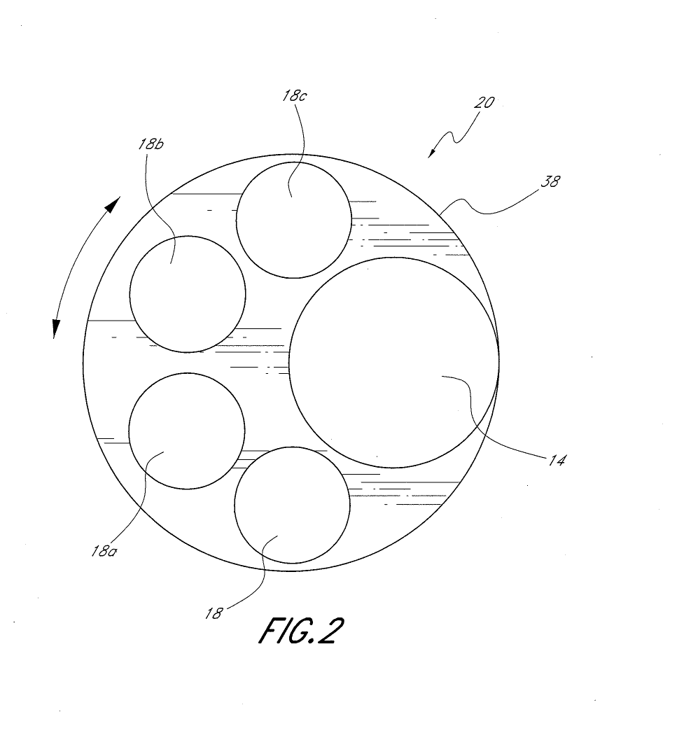

The dual lumen catheter includes a rotating dial guide 20 (FIG. 2). Ports 18, 18a, 18b, 18c of the rotating dial guide may be fixed or may rotate like a dial around port 14. Although 4 ports are shown, the dual lumen catheter can be adapted for any number of ports. The second lumen may include 1-10, 1-9, 1-8, 1-7, 1-6, 1-5, 1-4, 1-3, or 1-2 separate ports. In some embodiments, the second lumen may have more than 10 ports or apertures. The dial 38 is preferably calibrated in mm. The dial guide 20 can also be adapted to access other areas of the heart. The dial guide may be made of metal or plastic.

The catheter may also include guide wires and/or guide catheters and/or introducers to assist in placement. For example, a guide wire may be inserted, the sheath removed and exchanged for the cannula.

### Method

Embodiments of the method are directed to delivery of bioactive compositions, such as a platelet-containing composition, to the right side of the heart. The inventor has found that delivery to the right side of the heart provides more efficacious treatment of cardiac disorders such as myocardial infarction and arrhythmias.

In preferred embodiments, a method is employed such that the pathway to the pulmonary valve is first blocked off. That is the pathway from the superior vena cava to the right atrium to the right ventricle and then to the pulmonary valve is first blocked by use of flexible tubing, optionally in combination with a balloon. After blocking, a second device such as a catheter or cannula is introduced to access the right side of the heart, such as the coronary sinus.

With reference to FIG. 1, the distal portion 22 of the first cannula 22 is inserted into the right internal jugular vein. The first cannula 15 floats (following blood flow) into the right ventricle 34, toward the pulmonary valve 24. The balloon 30 just proximal to tricuspid valve 32 is inflated to block the tricuspid valve, thereby directing the second cannula to the coronary sinus 28. The second cannula 19 is inserted through the second port 18 through the second lumen 16 on the cephalad side of the first port 14. As access to the pulmonary valve is blocked by the first cannula, the second cannula is guided to the coronary sinus. Further guidance is provided by the rotating dial guide 20 further assisted by an imaging technique such as fluoroscopy. The second cannula 19 is directed toward the opening of the coronary sinus 28 by rotating the dial around the first cannula to position the second cannula, guided by the imaging means. Insertion of the second cannula 19 into the coronary sinus 28 is confirmed by fluoroscopy. The first cannula 15 acts as an anchor and guide for the second cannula 19.

The balloon 36 just proximal to tip of the second cannula 19 is inflated. Bioactive substance(s), such as unactivated Platelet Rich Plasma, is delivered through the second catheter 19. The balloon is maintained for up for 20-30 seconds and then released. Delivery may be repeated through the rotating dial guide 20 shown in FIG. 3. Repeat if needed using other ports 18a, 18b, 18c in a fixed manner.

In preferred embodiments, ports 18, 18a, 18b, 18c rotate around the first port 14. This rotation allows for precise placement of the second cannula 19. In an alternate embodiment, the ports may be fixed (not rotating). The second cannula 19 of the catheter may be removed from one port and moved to a second port selected from ports 18, 18a, 18b, 18c as needed until proper placement for access to the coronary sinus is achieved.

MRI, X-rays, trans-esophageal echocardiography or other external guidance means may be used to aid in placement of guide wires or catheters.

As is well known to those skilled in the art, catheters and cannulas may be introduced by use of guide catheters or introducers and guide wires. Typically, a guide catheter or introducer is introduced into an artery or vein which is made of a very soft very flexible material. A guide wire is then introduced through the guide catheter or introducer. The catheter or cannula is the introduced over the guide wire and the guide wire is removed. The guide catheter or introducer may stay in place or be removed. These techniques are well known to those skilled in the art. Such techniques may be employed to further assist one in placement of a catheter for accessing the coronary sinus as described in the method above.

It is not necessary to stop the heart in order to deliver the bioactive substance by use of the catheter as described above. However, in some embodiments, the beating heart may be stabilized during the delivery of the PRP composition. For example, in some variations, the beating heart may be slowed or stopped by delivery of one or more drugs and/or by electrical stimulation of the heart. For example, a heart may be stabilized using pharmacologic asystole. Alternatively or additionally, a heart may be stabilized using pacing or other algorithms that render the heart fairly static. These procedures may initiate various cardiac states such as reversible initiation of asystole, fibrillation, or a prolonged refractory state. In still other embodiments, mechanical stabilization of the cardiac tissue may be achieved using any of a variety of mechanical stabilizing systems. In some examples, a combination of stabilizing procedures may be used.

The PRP composition may be delivered during a specific portion of the cardiac cycle, and in these variations, the use of one or more stimulation electrodes to act as a pacemaker during the delivery may be desirable. For example, the beat-to-beat period may be artificially lengthened so as to deliver the PRP composition during a specific phase of the cardiac cycle. In these variations, the delivery device may include one or more stimulation and/or sensing electrodes. For example, sensing electrodes may be used to sense contractions of the heart, thereby allowing the delivery of composition to be timed with cardiac contractions. It may be desirable to deliver one or more components of the PRP composition between contractions of the heart.

In some examples, one or more cardiac sensors may be used during the treatment procedures. The sensors may be any suitable sensor system (e.g., an electrical sensor, a chemical sensor, a pressure sensor, an intravascular imaging sensor, or a biosensor) capable of detecting one or more signals indicative of a cardiac contraction or heartbeat. A cardiac sensor may be used to monitor the electrical activity of the heart by picking up and amplifying electrical signals from the heart and displaying a visual output and/or providing an audio output. For example, the output may be displayed on a display interface. The physician may use this output to inject the needles and/or composition into the tissue at a specific point in the cardiac cycle. The cardiac sensor may be coupled to a cardiac stimulator to manipulate or control the cardiac rhythm.

In some variations, a nerve stimulator may be used to electrically manipulate cardiac rhythm by stimulating the vagus nerve. Vagal stimulation may produce asystole (slowing or stopping of the heart). Once the vagal stimulation is stopped, the heart may return to a normal rhythm. Alternatively, the heart may be paced. Vagal stimulation, alone or in combination with electrical pacing, may be used selectively and intermittently to allow a physician to perform delivery of one or more components of the composition into a temporarily stopped heart.

Typically, vagal stimulation may slow or even prevent the heart from contracting. Following initial slowing or stopping of the heart, one or more components of the PRP composition may be delivered to the heart. Following a brief interval of nerve stimulation while the delivery may be performed, nerve stimulation may be ceased and the heart may be allowed to contract. A cardiac stimulator or pacemaker may be used to cause the heart to contract or the heart may be free to beat on its own. In some variations, one or more electrodes may be used for pacing the heart as desired. A processor may control both cardiac and nerve stimulation. For example, a processor may cease nerve stimulation and automatically begin cardiac stimulation.

Catheters other than that shown in FIGS. 1-2 may be used to deliver the PRP compositions. Transvascular delivery of compositions may comprise passing the delivery device through the coronary sinus into the cardiac venous system via the cardiac veins and, if needed, leaving the veins by tracking through myocardial tissue. Catheters may include one or more lumens and staggered or flush tips. The catheters may include needles or other devices (e.g., imaging devices) located at the distal end, and plungers or any other control located at the proximal end. The catheters and/or other delivery devices may have differently sized lumens to deliver multiple components of the PRP composition in the prescribed ratio. When catheters are used, a physician may navigate to the heart using one of the routes known for accessing the heart through the vasculature. Preferably, delivery is to the right side of the heart, more preferably via the coronary sinus.

The catheter for delivering the bioactive compositions may include cooled parts or other temperature control mechanisms to keep the bioactive composition at a desired temperature. Various embodiments of delivery devices may include a cooled chamber, and/or an agitator mechanism in a PRP chamber or injection chamber to prevent settling or clumping of the PRP components. For example, in some variations, the catheter has a cooled lumen or lumens for keeping the PRP composition cool during delivery. The delivery devices may additionally or alternatively include a mixing chamber for mixing the PRP composition prior to delivery. The PRP composition may also be stored in an agitating/vibrating chamber, or the physician may agitate the PRP composition once inside the delivery device by tilting or otherwise manipulating the device.

The total volume of the PRP composition delivered to the patient may be based on the size of the heart, the amount of the affected tissue, and/or the desired outcome of the procedure. For example, the total volume of composition injected may be less than 15000 μL.

The timing of PRP delivery relative to a cardiac dysfunction may be based on the severity of the cardiac dysfunction, the extent of the cardiac dysfunction, the condition of the patient, and the progression of any concurrent treatments. The PRP composition may be delivered at any suitable time. For example, it may be delivered immediately after the onset of an event such as myocardial infarction or an arrhythmia, within one hour of a myocardial infarction or an arrhythmia, one to eight hours following a myocardial infarction or an arrhythmia, or three to four days after a myocardial infarction or an arrhythmia after clinical stabilization of the patient when it is safer for the patient to undergo a separate procedure. The timing may be based upon the onset and/or the cessation of the cardiac dysfunction. In some variations, the composition is delivered about one week, about 1 to about 3 weeks, about 1 to about 6 months, or even up to or more than about 1 year after the onset of the cardiac dysfunction, particularly in the case of congestive heart failure and cardiac arrhythmia. Other times for treatment are also contemplated, including prior to any anticipated arrhythmia, and immediately upon finding an area of conductive tissue responsible for one or more arrhythmias (for preventing additional arrhythmias).

Alternatively or additionally, the bioactive composition may be used prophylactically, e.g., with certain conditions associated with an increased risk of cardiac dysfunction. For example, a PRP composition may be delivered one hour, thirty minutes, 15 minutes, 5 minutes, or just prior to or during a procedure associated with a heightened arrhythmia risk (e.g., a reperfusion procedure).

The PRP composition may be delivered at any suitable dose. In some embodiments, the dose may be between about 1 cc and about 3 cc, between about 3 cc and about 5 cc, between about 5 cc and about 10 cc, between about 10 cc and about 20 cc, or more. The dose may be delivered according to a medical procedure (e.g., at specific points in a procedure) and/or according to a schedule.

PRP composition may additionally or alternatively be used during procedures to correct congenital heart defects, or other pathologies. Examples of other cardiac procedures include, but are not limited to, angioplasty, coronary artery bypass, Minimally Invasive Direct Coronary Artery Bypass (MIDCAB), off-pump coronary artery bypass, Totally Endoscopic Coronary Artery Bypass (TECAB), aortic valve repair, aortic valve replacement, mitral valve repair, mitral valve replacement, Ross procedure, Bentall procedure, pulmonary thromboendarterectomy, valve-sparing aortic root replacement, cardiomyoplasty, Dor procedure, heart transplantation, septal myectomy, ventricular reduction, pericardiocentesis, pericardiectomy, atrial septostomy, Blalock-Taussig shunt procedure, Fontan procedure, Norwood procedure, Rastelli procedure, Maze procedure (Cox maze and minimaze), and/or pacemaker insertion. The PRP composition may used to prevent an arrhythmia associated with reperfusion of the cardiac tissue during any of the above procedures. As is known, reperfusion may cause a spontaneous arrhythmia to occur after cardiac surgery.

In some examples, a PRP composition may be used to treat a patient diagnosed with an acute myocardial infarction. Treatment with the PRP composition may occur in the field or in the emergency room setting. Criteria for PRP composition treatment may include positive cardiac markers, ST-elevations, or new wall motion abnormalities identified on echocardiogram, for example. The decision to treat with a PRP composition, and the treatment location(s), may depend upon one or more characteristics of the myocardial infarction. For example, a myocardial infarction may be characterized as a ST-elevation myocardial infarction (STEMI) or non-ST-elevation myocardial infarction (NSTEMI), a Q-wave or non-Q-wave myocardial infarction, and whether they are subendocardial or transmural. Myocardial infarctions may also be characterized anatomically by cardiac wall region and/or the suspected blockage site in the cardiac vasculature. Myocardial infarctions may also be characterized as anterior, lateral, inferior, posterior, septal, or right-ventricular in location, and may involve disease or blockage of the left-anterior descending, left circumflex, left main, posterior-descending and right coronary arteries, for example.

In other examples, timing of the PRP composition treatment may be based upon other treatments that are indicated in a patient with a myocardial infarction. In some instances, a PRP composition may be delivered before, during, and/or after reperfusion therapy is performed to treat an acute myocardial infarction or a previous myocardial infarction. Reperfusion therapies may include thrombolytic therapy, angioplasty, stenting (including bare metal stents and drug-eluting stents) or coronary artery bypass graft (CABG) surgery. In some instances, reperfusion therapy may be associated with an increased risk of an arrhythmia, including sudden death.

### Kits

A kit for performing the procedure described herein may include a catheter for accessing the right side of the heart, preferably the coronary sinus. In some embodiments the kit may include the catheter as described herein. However, the method is not limited to use with the described catheter. The kit may optionally include one or more preparation devices, one or more additional delivery devices, one or more collection devices, and/or instructions for use. The one or more preparation devices may be for preparing PRP and may comprise a centrifuge, for example. The one or more delivery devices may be configured to deliver a PRP composition comprising the PRP to a region of the heart to treat a cardiac dysfunction, preferably the right side of the heart, preferably the coronary sinus. The one or more collection devices may comprise one or more syringes, apheresis needles, or other devices for collecting blood from a patient. The patient may be presently suffering or have suffered a cardiac dysfunction. The components of the kit may be packaged in sterile containers. The kits may comprise one or more single-use components. Instructions may be in written or pictograph form, or may be on recorded media including audio tape, audio CD, video tape, DVD, CD-ROM, or the like.

The kits may be designed to target specific cardiac dysfunction such as myocardial infarction, cardiac arrhythmia or congestive heart failure. In one variation, a kit may be designed for use with a ventricular tachycardia. Such a kit may include, for example, one or more collection devices, ECG leads, and/or one or more anti-arrhythmic agents.

In addition to the foregoing uses for the compositions, methods and systems described herein, it will be apparent to those skilled in the art that other injured tissues, in addition to injured cardiac tissue, would benefit from the delivery of structural support materials to treat the injuries. Non-limiting examples of such tissues include the stomach, to reduce food intake and increase satiety; the abdominal wall, to prevent and treat hernias and the bladder to prevent or treat incontinence. Such tissues may additionally include vascular tissues.

### Examples

### Example 1

PRP was prepared using a centrifuge unit made by Harvest (Plymouth, Mass.). (Similar units are available as The Biomet GPS® system, the Depuy Symphony machine and the Arteriocyte Magellan® machine.) Approximately 55 cc of blood was drawn from the patient using a standard sterile syringe, combined with 5 cc of a citrate dextrose solution for anticoagulation, and then spun down to isolate the platelets according to the manufacturer's protocol. These platelets were then resuspended in approximately 3 cc of plasma. The resulting platelet rich plasma solution (PRP) was quite acidic and was neutralized with using approximately 0.05 cc of an 8.4% sodium bicarbonate buffer per cc of PRP under sterile conditions to approximately physiologic pH of 7.4. The PRP was not activated through addition of exogenous activators. This PRP composition is referred to herein as autologous platelet extract (APEX).

### Example 2

### Treatment of Acute Coronary Syndrome With PRP

A patient presents with symptoms of myocardial ischemia such as chest pain. The diagnostic evaluation including a physical exam, EKG, as well as laboratory studies determines that the patient is having acute coronary syndrome such as unstable angina, Non-ST elevation myocardial infarction, or ST elevation myocardial infarction. A blood sample is drawn to create platelet rich plasma. The patient is taken to the catheterization laboratory to perform reperfusion therapy and then have platelet rich plasma applied, injected, or instilled to improve cardiac rhythm or protect against reperfusion arrhythmia.

A dual lumen catheter, such as the catheter shown in FIGS. 1-2, is inserted into the right internal jugular vein. A first cannula 15 is allowed to float (following blood flow) into right ventricle 34. A guide wire is inserted, directed toward the pulmonary valve 24. The first cannula 15 is inserted and the balloon 30 around the first cannula 15 just proximal to tricuspid valve 32 is blown up. A second cannula 19 is inserted through port 18. This second cannula is directed toward the opening of the coronary sinus 28. A guide wire is inserted into the coronary sinus 28. Correct insertion may be confirmed by fluoroscopy. The second cannula 19 is inserted into the coronary sinus 28. A balloon 36 is blown up just proximal to tip of the second cannula 19. The platelet-rich plasma (or bioactive composition) is delivered. The balloon is released after 20-30 seconds. This procedure may be repeated using additional ports (18a, 18b, 18c) as needed. Rotation around the first port 14 allows for precise placement of the second guide wire.

MRI, X-rays, trans-esophageal echocardiography or other external guidance means may be used to aid in placement of guide wires or catheters.

The PRP in the above example can be prepared as described in Example 1 or alternatively using techniques including, but not limited to, centrifuges, gravity filtration devices, cell sorting or others. It can be combined with stem cells, genetic engineering or mechanical devices such as permanent or bioaborbable pacemaker or stent. The PRP can be autologous or made from allogenic sources.

### Example 3

### Treatment of Arrhythmia Using the Method From Example 2

A patient presents with symptoms of palpitations, lightheadedness and pre-syncope or syncope. A diagnostic evaluation including physical exam and EKG determines that the patient is in a sustained or non-sustained arrhythmia such as, but not limited to, supraventricular or ventricular tachycardia. The patient is treated at the bedside or taken to the catheterization laboratory to have PRP injected or instilled into the location of the arrhythmia as located by topographic electrocardiogram or catheter-based electrophysiology study as discussed in Example 2 above.

### Example 4

### Treatment of Reperfusion Arrhythmia Using the Method From Example 2

PRP can be used to prevent arrhythmia associated with reperfusion in ischemic myocardial tissue. A patient who is undergoing cardiac surgery requiring bypass support such as, but not limited to, coronary artery bypass grafting, valve repair, valve replacement, cardiac transplantation, or other cardiac surgeries can have PRP injected or instilled into the myocardial tissue prior to, during, or after reperfusion. Reperfusion occurs when coming off the bypass machine. Injection is performed as described for Example 2. The PRP is administered via single or multiple injections.

While methods, devices, and kits have been described in some detail here by way of illustration and example, such illustration and example may be for purposes of clarity of understanding only. It will be readily apparent to those of ordinary skill in the art in light of the teachings herein that certain changes and modifications may be made thereto without departing from the spirit and scope of the appended claims.

What is claimed is:

1. A method of delivering bioactive composition to heart comprising:
inserting a first cannula of a dual lumen catheter into a vein to access the right ventricle, wherein the first cannula comprises a first balloon;
blowing up the balloon around the first cannula proximal to the tricuspid valve;
inserting a second cannula of the dual lumen catheter comprising a second balloon into the coronary sinus;
blowing up the second balloon proximal to the tip of the second cannula; and
delivering the bioactive composition to the coronary sinus.

2. The method of claim 1, wherein the bioactive composition comprises platelet-rich plasma.

3. The method of claim 2, wherein the bioactive composition does not include an activator of platelet-rich plasma.

4. The method of claim 2, wherein the platelet-rich plasma composition is buffered to a pH of 7.3 to 7.5.

5. The method of claim 2, wherein the platelet-rich plasma composition comprises platelets obtained from the patient.

6. The method of claim 2, wherein the platelet-rich composition comprises 10-50% of neutrophils compared to whole blood.

7. The method of claim 2, wherein the platelet-rich composition comprises white blood cells at a level of 3-5 times higher than baseline.

8. The method of claim 1, wherein the vein is in the neck or groin.

9. The method of claim 8, wherein the vein is the right internal jugular vein.

10. A dual lumen catheter comprising a first lumen comprising a first port for accessing the pulmonary valve and a second lumen comprising a second port for accessing the coronary sinus, wherein the first lumen and second lumen comprise a dial guide.

11. The dual lumen catheter of claim 10, wherein the second port comprises 1-5 apertures.

12. The dual lumen catheter of claim 11, wherein the apertures of the second port rotate around the first port.

13. A method of delivering a composition comprising platelet-rich plasma to heart muscle comprising delivering the composition comprising platelet-rich plasma to the right side of the heart.

14. The method of claim 13, wherein the composition does not include an activator of platelet-rich plasma.

15. The method of claim 13, wherein the platelet-rich plasma composition is buffered to a pH of 7.3 to 7.5.

16. The method of claim 13, wherein the platelet-rich plasma composition comprises platelets obtained from the patient.

17. The method of claim 13, wherein the platelet-rich composition comprises 10-50% of neutrophils compared to whole blood.

18. The method of claim 13, wherein the platelet-rich composition comprises white blood cells at a level of 3-5 times higher than baseline.

19. The method of claim 13, wherein the delivery is through a vein.

20. The method of claim 19, wherein the vein is the right internal jugular vein.
